(19) United States
(12) Plant Patent Application Publication
(10) Pub. No.: US 2002/0194664 P1
(43) Pub. Date: Dec. 19, 2002

(54) Hemerocallis hybrid named 'Malja'

(76) Inventor: Jacques Deetlefs Malan, Rosslyn (ZA)

Correspondence Address: Jacques Malan, Botanical Department, Monrovia Nursery, 18331 E. Foothill Blvd., Azusa, CA, 91702, US

(21) Appl. No.: 09/880,924

(22) Filed: Jun. 15, 2001

(51) Int. Cl. A01H 5/00

(52) U.S. Cl. PLT/312

(57) ABSTRACT

A new and distinct selection of Hemerocallis hybrid cultivar ‘Malja’ characterized by its unique combinations of yellow to white and green foliage variegation and golden yellow flowers that are borne on short stems about half the height of the foliage on a plant of short stature.

This invention relates to a new and distinct selection of Hemerocallis hybrid, which is native to Europe, China and Japan, and a member of the Lily family. The new variety, Hemerocallis hybrid cv. ‘Malja’ resulted from seed which was sown in 1989 of Hemerocallis Miniature Hybrids. These seeds characteristically produced plants with a small growth habit. In 1990 a seedling was discovered in South Africa with yellow and green variegation and with dark yellow flowers on short stems. This new selection will be marketed as a perennial landscape plant. ‘Malja’ can be grown in shade as well as full sun and is slow growing and therefore ideal as a patio or container plant with its unusual variegated foliage.

The new plant has been asexually reproduced by divisions since its discovery in 1990 in South Africa and since 1995 in Azusa, California. Through extensive asexual propagation of this plant and observation, it has been established that the plant is true to type and the novel, exceptional characteristics of this new plant are fixed and stable and reliably passed on to progeny through asexual reproduction.

### SUMMARY

Hemerocallis hybrid cultivar ‘Malja’ possesses a vibrant foliage variegation. The foliage in shade is green in the central area of the leaf and white on the margins. In sun conditions the white leaf color turns to a cream yellow color.

Typically, Hemerocallis plants range in height of two to three feet. The growth habit of ‘Malja’ remains in the 12″ to 15″ range. The cultivar ‘Malja’ typically will bloom for two to three months of the year in South Africa and in Southern California.

### BRIEF DESCRIPTION OF THE DRAWING

FIG. 1 photograph exhibits a close up of the flower and foliage of cultivar ‘Malja’. FIG. 2 photograph shows the growth habit and variegation of cultivar ‘Malja’ and the flowers that appear on short stems within the center of the plant.

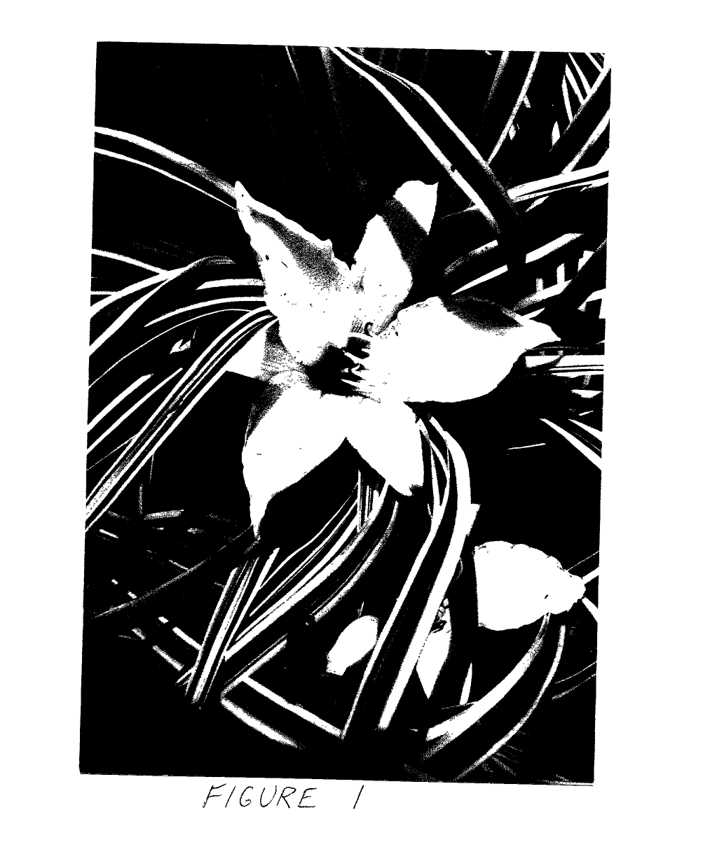

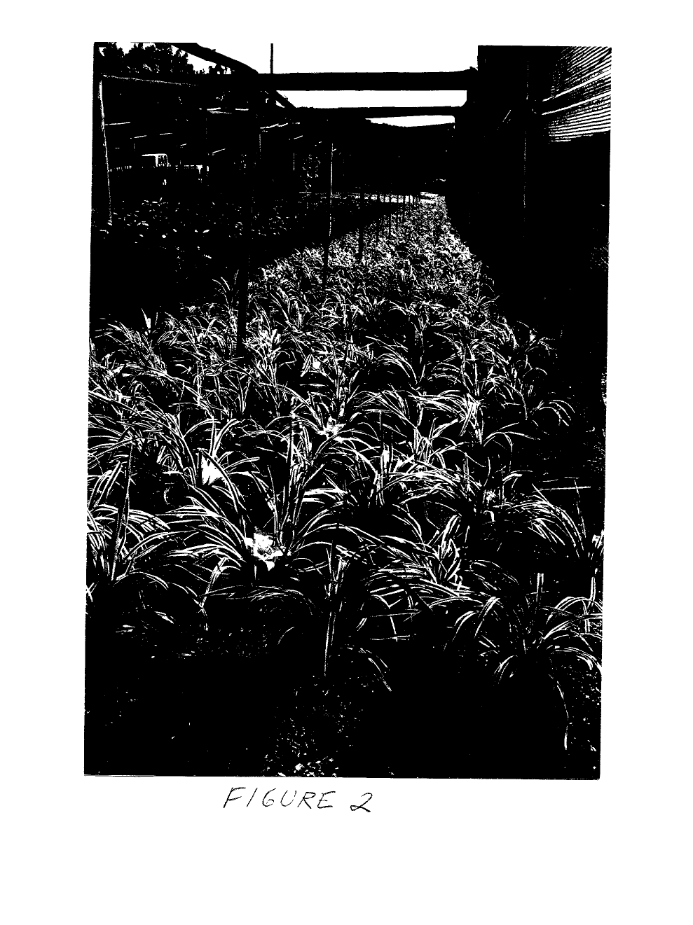

### BOTANICAL DESCRIPTION

Below is a detailed description of Hemerocallis hybrid ‘Malja’. Color terminology is from The Royal Horticultural Society Colour Charts. Measurements are based on plants grown in South Africa and Southern California.

Overall habit: herbaceous perennial

Overall size:

Maximum height.—from soil surface to top of foliage ranges from 30 cm to 38 cm

Maximum width.—of the plant ranges from 30 cm to 45 cm

Flowering time in Southern California: flowering commences in mid May and continues until July

Flowering time in South Africa: flowering commences in mid November (summer in Southern Hemisphere) and continues until early January

Dormancy: winter dormant

### FLOWER

Arrangement: in clusters of up to 7 on short scapes. Position of flower at time of bloom is approximately half the height of the foliage.

Type: perianth composed of six similar segments, funnel form to wide—flaring.

Diameter: to 12 centimeters

Color: uniform on inner and outer portion of perianth segments Yellow-Orange Group 17B

Texture of perianth segments: slightly fleshy

Duration of bloom: flowers ephemeral

Perianth margin: slightly undulate

### REPRODUCTIVE STRUCTURES

Stamens: 6 in number

Anthers.—Color: Greyed-Orange Group 166A Length: averaging 4 mm Width: averaging 1 mm

Filaments.—Color: Yellow-Orange Group 17B Length: averaging 2 cm Width: less than 2 mm

Pistil: Color: Yellow-Orange Group 17B Length: averaging 3 cm

Fruit: to date not observed

### FOLIAGE

Size: Length: ranges from 40 cm-52 cm Width: ranges from 1.5 cm-2.5 cm

Shape: Overall: linear, long, strap-like, keeled leaves Tip: narrow, acuminate tip

Margin: entire, slightly to strongly undulate

Color: variegation color varies in sun and shade exposure, typically more yellow is exhibited in full sun conditions and more white is exhibited in shade conditions. Center of leaf is green and margins are variegated.

Leaf center color.—Yellow-Green Group 146A to Yellow-Green Group 147C. Green portion is fairly uniform varying from 5 mm to 10 mm in width and comprising over half the width of the leaf in this color.

Leaf margin color.—Yellow Group 4C to Yellow Group 7C. The yellow to white variegation exists the length of the entire leaf and on either side of the central green portion of the leaf. The variegation width varies from 5 mm to 10 mm in width.

Texture.—thin, coriaceus

Arrangement: arising from base of plant

Pest/Disease Resistance: similar to other Hemerocallis plants, susceptible to red spider mites.

Heat/Drought Tolerance: prefers temperatures 70° F. to 100° F. with ample soil moisture. Mature plantings tolerate dry soil conditions.

I claim:

1. A new and distinct Hemerocallis plant substantially as herein shown and described.
